(19) United States
(12) Patent Application Publication
(10) Pub. No.: US 2024/0065839 A1
(43) Pub. Date: Feb. 29, 2024

(54) BIOMIMETIC METAMATERIAL SLEEVES FOR EXTERNAL SUPPORT OF HUMAN VENTRICLE(S) AND METHODS FOR MAKING AND USING THEM

(71) Applicant: THE BOARD OF TRUSTEES OF THE LELAND STANFORD JUNIOR UNIVERSITY, Stanford, CA (US)

(72) Inventors: Ileana Pirozzi, Palo Alto, CA (US); Ali Kight, Palo Alto, CA (US); Kyung Won Han, Stanford, CA (US); Rohan Shad Arora, San Jose, CA (US)

(21) Appl. No.: 18/375,498

(22) Filed: Sep. 30, 2023

Related U.S. Application Data
(63) Continuation of application No. PCT/US22/23198, filed on Apr. 2, 2022.
(60) Provisional application No. 63/278,432, filed on Nov. 11, 2021.
(60) Provisional application No. 63/170,443, filed on Apr. 2, 2021.

(51) Int. Cl. A61F 2/24

(52) U.S. Cl. CPC A61F 2/2481; A61F 2210/0057; A61F 2210/0076; A61F 2230/0019; A61F 2240/007; A61F 2240/008; A61F 2250/0009; A61F 2250/0012; A61F 2250/0018; A61F 2250/0028; A61F 2250/0048; A61F 2250/0096

(57) ABSTRACT

Devices and methods are provided for supporting a subject's heart, e.g., to support the left and/or right ventricles of the heart, e.g., to prevent and/or treat heart failure. In one example, the device includes a sleeve configured to be implanted over a region of the subject's heart including a lattice formed on a surface of the sleeve. The sleeve may be positioned and permanently implanted over a desired region of the subject's heart to provide passive support to ventricular tissue of the heart.

### RELATED APPLICATION DATA

The present application is a continuation of International Application No. PCT/US2022/023198, filed Apr. 2, 2022, which claims benefit of U.S. provisional application Ser. No. 63/170,443, filed Apr. 2, 2021, and 63/278,432, filed Nov. 11, 2021, the entire disclosures of which are expressly incorporated by reference.

### STATEMENT REGARDING FEDERALLY SPONSORED RESEARCH AND DEVELOPMENT

This invention was made with Government support under contract GRFP (Ali Kight) awarded by the National Science Foundation. The Government has certain rights in the invention.

### TECHNICAL FIELD

The present application relates to medical devices and, more particularly, to devices for supporting a subject's heart, e.g., to support the left and/or right ventricles of the heart, e.g., to prevent and/or treat heart failure, to methods for making such devices, and to systems and methods for implanting and using such devices.

### BACKGROUND

Alterations to the complex biomechanics of the heart are critical factors in the onset and development of ventricular dysfunction and ultimately heart failure. Thus, restoration and regulation of tissue deformation is critical in supporting a disadvantaged heart. Nevertheless, to date, no technologies have been deployed clinically for the regulation of regional tissue mechanics. Further, devices that do aim to support the disadvantaged heart are blood contacting, creating a high risk of thrombotic events.

The modern-day heart failure epidemic sees two conflicting trends: as the number of patients who develop progressive heart failure continues to rise, the number of available transplantable hearts has remained relatively constant. This mismatch in clinical demand and supply has been largely responsible for the rapid rise in left ventricular assist device (“LVAD”) utilization in patients with advanced heart failure. Yet, LVADs incur several highly unpredictable and often fatal complications such as right heart failure (“RHF”), which occurs in as many as 30-40% of patients shortly after LVAD implantation, portending increased morbidity and mortality. In the period following LVAD insertion, the right ventricle (“RV”) is challenged in a number of ways. First, with near normalization of left ventricle (“LV”) output, RV filling increases dramatically. Second, the LVAD causes mechanical unloading of the ventricular chamber, resulting in geometrical changes in the RV. Third, increases in vascular resistance encountered further compromise ventricle function.

The clinical challenge is exacerbated by the lack of reliable means to accurately predict chances of RHF at the time of implantation. After clinical manifestation of RHF, surgeons are currently faced with two options: (1) a secondary operation, exposing the recovering patient to high perioperative risk, in order to implant a second LVAD as an “RVAD” on the right ventricle (note: this is an off-label use of the device, due to lack of a better alternative), or (2) avoid a second operation, due to precarious patient conditions, and rely on other highly ineffective treatment strategies (such as ECMO or pharmacologicals). Studies have shown that early implantation of “RVAD” devices, at the time of LVAD implantation demonstrates superior outcomes. However, without the ability to reliably predict which patient will develop postoperative RHF, surgeons have to balance the risks of delayed RV support, with the inherent risk of the VAD devices themselves—VADs are blood contacting, prone to thromboembolic events and confer an estimated 3-fold increase in risk of death.

Therefore, there is a need for cardiac support devices to optimize ventricular tissue mechanics in heart failure patients.

### SUMMARY

The present application is directed to medical devices and, more particularly, to devices for supporting a subject's heart, e.g., to support the left and/or right ventricles of the heart, e.g., to prevent and/or treat heart failure, to methods for making such devices, and to systems and methods for implanting and using such devices.

For decades, the focus of heart failure treatment and interventions has largely been the left ventricle, with one of the most groundbreaking technologies added to the toolkit of heart failure treatment being the Left Ventricular Assist Device (LVAD). In 2020, the US Ventricular Assist Device (“VAD”) market was estimated to be $1.3 B, with demand and adoption doomed to rise due to increased incidence of cardiovascular disease and favorable reimbursement policies. Yet, while LVADs have greatly improved outcomes in patients with late-stage heart failure, right heart failure following implantation remains a highly unpredictable, often fatal complication. To date, up to 40% of LVAD patients develop right heart failure (RHF) shortly after implantation, facing a paucity of clinical management options.

The devices herein may provide non-blood contacting, mechanically responsive LVAD-adjunct solutions to address the deterioration of RV function, e.g., in the context of late-stage heart failure. A device of this safety profile may be prophylactically implanted, e.g., at the time of primary LVAD implantation for patients, to provide desired epicardial restraint, which may obviate the need to plan for secondary procedures or rely on substandard predictive models for RHF. With an eye on clinical translation, the devices may provide passive support to allow for targeted rehabilitation of the heart. The potential for functional recovery is vested in the idea that mechanical factors are important stimuli in normal and pathologic physiology, particularly in tissues that are essentially mechanical in nature. Thus, the devices herein may provide manipulation of the mechanical environment of the right heart and/or offer therapeutic benefits in a high-need clinical setting, yielding the first-of-its-kind LVAD-adjunct technology targeting the right heart.

In one example, a passive sleeve is provided that includes a metamaterial lattice composed of soft, flexible material or multi-materials. The lattice structure of the device may include various unit cell geometries that, for example, exhibit desired anisotropic or auxetic properties. The density of the unit cells of the lattice may be spatially varied to tune heterogeneous effective stiffness and thus program deformation patterns. Overall, the deformation patterns of the sleeve may be tuned to match healthy, physiologic ventricular tissue mechanics, derived from computational modeling and patient imaging data.

The devices herein may lead to the development of several implantable surgical devices for the prevention and treatment of heart failure. For example, the devices may be used to support the left ventricle in the presence of ischemic cardiomyopathy. Additionally, the devices may be co-implanted with left ventricular assist devices (LVADs) to support both ventricles and/or the right ventricle. Right heart failure is a common, yet unpredictable, complication of LVAD implantation, as the right ventricle has to support an increased volume of blood. The devices herein may be used as a prophylactic, passive support that aids ventricular mechanical function. This application would fit into the current clinical workflow, may work synergistically with existing devices on the market (see LVADs), and/or may cause no additional risk due to the non-blood contacting nature of the devices.

In accordance with one example, a device is provided for supporting a subject's heart that includes a sleeve configured to be implanted over a region of the subject's heart comprising a lattice defining a surface of the sleeve.

In accordance with another example, a passive device is provided for supporting a subject's heart that includes a sleeve comprising an open upper end and a lower end defining an interior region sized to receive a portion of the heart, the sleeve comprising a plurality of elongate elements members coupled together to define a plurality of interconnected cells configured to provide epicardial support of the heart.

In accordance with still another example, a passive device is provided for supporting a subject's heart that includes a sleeve comprising an open upper end and a lower end defining an interior region sized to receive a portion of the heart, the sleeve comprising a plurality of elongate elements members coupled together at their ends to define a plurality of interconnected cells surrounding open regions configured to provide epicardial support of the heart.

In accordance with another example, a method is provided for designing a device for supporting a subject's heart that includes generating a desired deformation map from imaging data of a heart; designing cell parameters for a lattice of the device based at least in part on the deformation map; forming the device including the lattice from elastic material include cells comprising the cell parameters; fitting the support device around a phantom heart; actuating the phantom heart to simulate pulsation of the heart while monitoring deformation of the support device; and comparing actual deformation of the support device to the desired deformation map.

In accordance with yet another example, a method is provided for making a device for supporting a subject's heart that includes generating a desired deformation map from imaging data of a heart; designing cell parameters for a lattice of the device based at least in part on the deformation map; and forming a sleeve comprising an open upper end and a lower end defining an interior region sized to receive a portion of the heart, the sleeve comprising a plurality of elongate elements members coupled together to define a plurality of interconnected cells comprising the cell parameters.

In accordance with another example, a method is provided for supporting a subject's heart that includes positioning a sleeve over a region of the heart, the sleeve comprising lattice formed on a surface of the sleeve configured to provide passive support to ventricular tissue of the heart.

In accordance with still another example, a method is provided for supporting a subject's heart that includes providing a sleeve comprising an open upper end and a lower end, the sleeve comprising a plurality of elongate elements members coupled together to define a plurality of interconnected cells; and positioning the sleeve over a region of the heart to provide passive support.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features and design elements of the drawings are not to-scale. On the contrary, the dimensions of the various features and design elements are arbitrarily expanded or reduced for clarity. Included in the drawings are the following figures.

FIG. 1 shows an example of a passive sleeve implanted over a portion of a subject's heart.

FIGS. 2A and 2B show a first example of a cell configuration, including diamond-shaped cells, that may be provided in a passive sleeve, showing the cells of the lattice changing shape during a cardiac cycle, e.g., at the end of systole (FIG. 2A) and at the diastole (FIG. 2B).

FIGS. 3A and 3B show a second example of a cell configuration, including bowtie-shaped cells, that may be provided in a passive sleeve, showing the cells of the lattice changing shape during a cardiac cycle, e.g., at the end of systole (FIG. 3A) and at the diastole (FIG. 3B).

FIGS. 3C and 3D are details showing axial deformation of cells of the sleeve shown in FIGS. 3A and 3B.

FIG. 3E is a graph showing desired Poisson and relative stiffness ratios as a function of RV region, i.e., base, mid-cavity, and apex, displayed as arbitrary units (AU).

FIG. 3F is a table showing examples of geometrical parameters and associated variables of an exemplary bowtie cell.

FIGS. 4A and 4B show a third example of a cell configuration, including sinusoidal-shaped cells, that may be provided in a passive sleeve, showing the cells of the lattice changing shape during a cardiac cycle, e.g., at the end of systole (FIG. 4A) and at the diastole (FIG. 4B).

FIG. 5 shows a fourth example of a cell configuration, including interconnected sinusoidal-shaped cells, that may be provided in a passive sleeve.

FIG. 6 is a flow chart showing an exemplary design process for making a sleeve device, such as the device shown in FIG. 1.

FIGS. 7A and 7B show an example of a sleeve device including an integral strain sensor array.

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is well established that biomechanical stress and strain concentrations due to chronically increased hemodynamic demand cause adverse growth (or “remodeling”) of cardiac tissue culminating in progressive ventricular dysfunction and eventually failure. Studies have demonstrated that optimized epicardial restraint has beneficial effects on normalizing ventricular wall stress and in turn attenuating the risk of remodeling. Importantly, these studies have confirmed the need for epicardial devices to 1) be preferentially tuned with respect to the significant difference in the optimal mechanical restraint requirements between the LV and RV, and 2) recapitulate native tissue mechanics so as to ensure healthy deformation patterns. In particular, auxetic elements (i.e., structures that exhibit a negative Poisson ratio) have demonstrated efficacy in restoring healthy mechanical behavior in cardiac tissue.

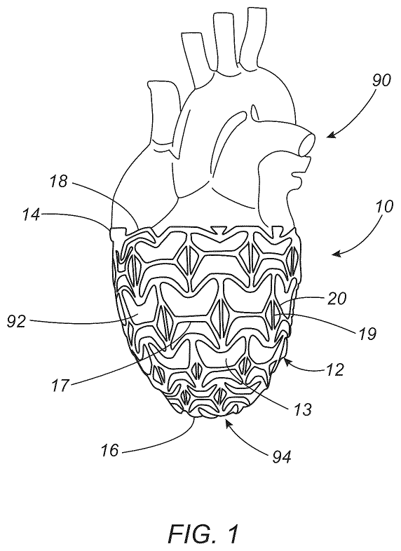

Turning to the drawings, FIG. 1 shows an example of device 10 including a fully passive, elastic support lattice designed to be co-implanted prophylactically over at least a portion of a subject's heart 90, e.g., at the time of LVAD-implantation. An individual device may be designed and constructed based on an individual patient's anatomy, or, alternatively, the lattice structure may be developed in multiple sizes and/or shapes to fit the majority of human hearts, as described further elsewhere herein. For example, the device 10 herein may be configured to act as a mechanical template, e.g., to regionally vary mechanical behavior of a right ventricle (“RV”), such that the device 10 may bias tissue deformation of the heart 90 towards healthy patterns, e.g., when placed on a diseased ventricle. Specifically, deformation behavior of the device 10 may be configured to match spatially varying RV tissue stiffness and Poisson ratios (defined herein as the lateral extension or compression of a material under axial stretch).

In one example, the device 10 may include a flexible lattice patterned with auxetic motifs designed to provide programmed mechanical behavior and/or achieve anisotropic mechanical properties typical of native heart tissue, e.g., as derived from computational modeling and/or imaging data. The device 10 may provide passive support to ventricular tissue, e.g., by (a) providing mechanical offloading to counter the effects of increased preload on the ventricular wall, and/or (b) redistributing biomechanical loads based on optimal ventricular deformation maps. Thus, the device 10 (and other devices herein) may provide simple and effective manipulation of the mechanical environment, e.g., of the right heart, through rationally programmed heterogeneity and may offer therapeutic benefits in a high-need clinical setting.

Generally, as shown in FIG. 1, the device 10 includes a plurality of struts or elements 20 coupled together, e.g., in one more cell configurations, to define a body or sleeve 12 including an open upper end 14, a closed and/or relatively narrow lower end 16, and a plurality of open regions 13 within the cells defined by the elements 20, e.g., extending entirely through the sleeve 12. The elements 20 may surround an interior region 18 of the sleeve 12 sized to receive a portion of a patient's heart 90, e.g., covering one or both ventricles and extending to the apex 94 of the heart 90, as shown in FIG. 1. In the example shown in FIG. 1, in its relaxed state, the sleeve 12 may define a partial ovoid or other three-dimensional shape, e.g., such that the sleeve 12 expands partially from the upper end 14 before tapering down to the lower end 16. Alternatively, the sleeve 12 may define a generally conical shape tapering from the upper end 14 inwardly towards the lower end 16, or may define other desired shapes corresponding to the anatomy received within the interior 18.

In the example shown, the sleeve 12 may be sized such that the lower end 16 surrounds and/or engages the apex 94 of the heart 90, and the upper end 14 is positioned over the epicardium 92 surrounding and/or above the right ventricle (not shown) within the heart 90. The sleeve 12 may be sufficiently flexible to allow the sleeve 12 to expand and contract circumferentially and/or otherwise (e.g., expanding and contracting simultaneously both longitudinally and circumferentially) to increase in size to accommodate expansion and contraction of the heart 90 during the cardiac cycle, while continuing to support the heart 90. The sleeve 12 may include a substantially uniform thickness (i.e., between the outer surface 17 and the inner surface 19 of the sleeve 12) or may have different thicknesses in different regions, if desired.

In one example, the elements 20 may be integrally formed together to define a plurality of cells with open regions 13 between the elements 20, e.g., such that the elements 20 define the entirety of the sleeve 12. For example, the device 10 may be created, e.g. by molding, casting, 3D printing, and the like, to provide an interconnected array of elements 20, optionally defining a variety of cell configurations overlying different regions of the heart 90. Alternatively, a solid-walled body defining the upper and lower ends 14, 16 may be formed, e.g., by molding, casting, 3D printing, and the like, and then the open regions 13 and resulting elements 20 may be formed by removing material, e.g., by laser cutting, machining, etching, and the like. Alternatively, cells or other subsets of elements 20 may be formed separately, e.g., individually or in desired linear arrays or other sets, which may be attached together, e.g., by one or more of bonding with adhesive, laser welding, fusing, suturing, and the like, to provide the sleeve 12.

The sleeve 12 may be formed from one or more biocompatible materials, e.g., polymers, such as polyurethane, elastomeric materials, such as silicone, and the like, that provide the desired mechanical characteristics for supporting the heart 90. For example, the elements 20 defining the entire sleeve 12 may be formed entirely from flexible materials having a shore hardness between about 70-90 A, e.g., having anisotropic and/or auxetic properties in one or more regions of the sleeve 12, e.g., as described elsewhere herein. Alternatively, additional materials may be embedded in or otherwise attached to the elements 12 to enhance and/or provide desired mechanical properties in one or more regions of the body 12. For example, elastic elements, e.g., elastic or superelastic wires formed from Nitinol or other metal, plastic, or composite materials (not shown) may be embedded within the elements 20 to enhance or otherwise modify the mechanical properties of the resulting lattice of the sleeve 12.

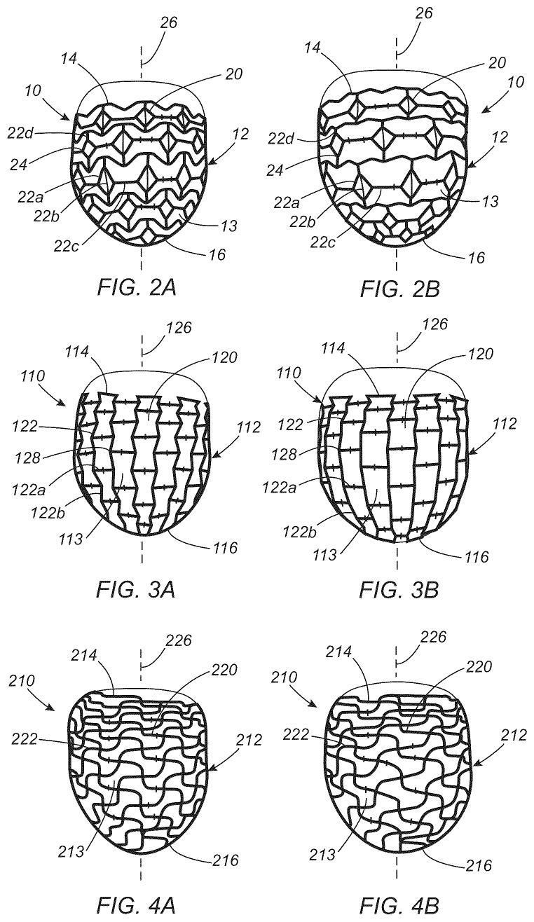

Turning to FIGS. 2A and 2B, an example of a sleeve 10 is shown that includes a plurality of diamond-shaped cells 22 defined by elements 20. As shown, each diamond-shaped cell 22 may include four diagonal elements 20a connected together at each of their ends to adjacent elements 20, i.e., at apices 24. Optionally, at least some of the cells may include an additional element 20b extending between opposite apices 24, e.g., aligned with a longitudinal axis 26 extending between the upper and lower ends 14, 16 of the sleeve 12. The cells 22 may be arranged in a desired pattern, e.g., in circumferential rows orthogonal to the axis 26 around the sleeve 12 with adjacent cells 22 connected by additional circumferential elements 20c. Axially adjacent rows of cells 22 may be interconnected, e.g., by curved elements 20d interconnected between upper and lower apices 24a, 24b such that the resulting sleeve has anisotropic mechanical properties. For example, the axially adjacent diamond-shaped cells 22 may be offset diagonally from one another and interconnected by the curved elements 20d.

The configuration of the cells 22 may be substantially uniform in the sleeve 12 or may be varied, e.g., in size, shape, and/or spacing, to customize the mechanical support, e.g., axially between the upper and lower ends 14, 16 and/or around the circumference of the sleeve 12. Alternatively, the size, shape, and/or spacing of the cells 22 may be modified in different regions of the sleeve 12, e.g., to enhance support and/or further model proper movement of the heart 90. In addition or alternatively, the width of different elements 20 (i.e., the dimension along the surface of the sleeve 12, e.g., axially and/or circumferentially) may be modified to further program the mechanical properties of the sleeve 12 in a desired manner, e.g., as described elsewhere herein.

The resulting cells 22 may be auxetic such that expansion and/or elongation of the sleeve 12 along the axis 26, e.g., at the end of diastole or otherwise during the cardiac cycle, causes the curved elements 20d to at least partially straighten, e.g., as shown in FIG. 2B. The elements 20 may be biased to return towards a more relaxed configuration, such as that shown in FIG. 2A, which may enhance restraint of the epicardium to support the heart, e.g., to normalize ventricular wall stress and in turn attenuating the risk of remodeling, particularly of the right ventricle, as described further elsewhere herein.

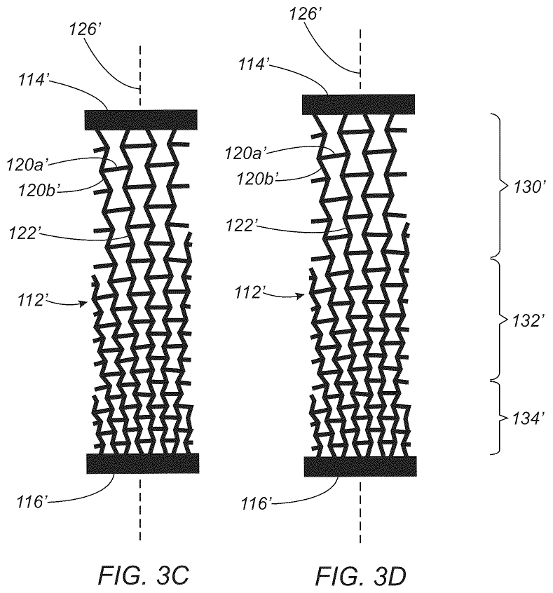

Turning to FIGS. 3A-3D, another example of a device 110 is shown that includes a sleeve 112 generally constructed similar to the previous device 10, e.g., principally or entirely from a plurality of elongate elements 120 that are interconnected with one another define a plurality of cells 122 surrounding respective open regions 113. Unlike the previous device 10, the cells 122 include a plurality of elements 120 that are interconnected to define open regions 113 having a reentrant honeycomb (referred to herein as “bowtie”) or hourglass shape.

For example, with additional reference to FIG. 3F, each bowtie-shaped cell 122 may include first and second substantially straight elements 120a opposite one another, and first and second angled elements 120b extending between opposite ends 124, respectively, of the first and second substantially straight elements 120a. The angled elements 120b may taper inwardly towards one another at intermediate regions 128 between the opposite ends 124 to define the bowtie-shape. For example, the angled elements 120b may include substantially straight sub-elements 120c that extend at acute angles relative to the first and second straight elements 120a and connect at the intermediate regions 128. Alternatively, the angled elements 120b may have curved or other shapes with a convex side defining the narrow intermediate region of the open region 113 of each cell 122.

In the example shown in FIGS. 3A-3D, the first and second straight elements 120a extend generally around the circumference of the sleeve 112, e.g., substantially perpendicular to the longitudinal axis 126 (which may be generally aligned along the surface of the sleeve 112 between the upper and lower ends 114, 166 to provide a reference frame for the surface), and the angled elements 120b may be aligned generally along the axis 126 (although not truly parallel given their angled shape).

In addition, the cells 122 may be interconnected such that elements 120 may define a portion of multiple open regions 113, e.g., an upper straight element 120a of one cell 122 may also be the lower straight element 120a of an axially adjacent cell 122. Circumferentially adjacent cells 122 may be offset laterally from one another, e.g., such that the angled elements 120b of one cell 122 may define portions of angled elements 120b of two laterally adjacent cells 122.

The resulting array of elements 120 may be configured to support the heart in multiple directions, e.g., vertically and/or horizontally along the surface of the heart 90. For example, the bowtie-shaped cells 122 may be auxetic such that expansion and/or elongation of the sleeve 112 along the longitudinal axis 126, e.g., at the end of diastole or otherwise during the cardiac cycle, causes the angled elements 120b to at least partially straighten, e.g., as shown in FIG. 3B. The elements 120 may be biased to return towards a more relaxed configuration, such as that shown in FIG. 3A, which may enhance restraint of the epicardium to support the heart, e.g., to normalize ventricular wall stress and in turn attenuating the risk of remodeling, particularly of the right ventricle, as described further elsewhere herein.

The auxetic properties of the bowtie-shaped cells 122 is demonstrated in FIGS. 3C and 3D, which show elongation of a sleeve 112′ (generally similar to sleeve 112) along axis 126,′ which may cause the elements 120′ to thicken laterally, e.g., substantially perpendicular to the axis 126.′

As shown in FIGS. 3C and 3D, the sleeve 112′ may include different regions having different size cells 122.′ For example, a first or upper circumferential region 130′ adjacent the upper end 114′ may include cells 122′ that have an axial length and/lateral width (e.g., perpendicular to the axis 126′) that are larger than a second or central circumferential region 132′. The central region 132′ may, in turn, include cells 122′ that have an axial length and/lateral width that are larger than a third or lower circumferential region 134′ adjacent the lower end 116.′ As shown, the size of the cells 122′ may gradually decrease from the upper end 114′ to the lower end 116,′ although alternatively, the different regions may have substantially uniform size cells that transition between the regions (not shown). Optionally, the width of the cells may be otherwise varied, e.g., around the circumference of the sleeve 112′ in addition to varying the size in different axial regions, e.g., to provide a device 110′ that is programmed to support different regions of the heart in a desired manner, e.g., as described further elsewhere herein.

For example, FIG. 3E shows exemplary relative desired Poisson and relative stiffness ratios that may be provided in different regions of a sleeve as a function of RV region of the heart. For example, it may be desirable to have a lower Poisson ratio and stiffness in a central region (e.g., region 132′ in FIGS. 3C and 3D) corresponding to the mid-cavity of the RV, than at an upper region (e.g., region 130′) corresponding to the base of the RV and a lower region (e.g., region 134′) corresponding to the apex of the RV. FIG. 3F shows geometrical parameters and associated variables of an exemplary bowtie cell 122 that may be modified, e.g., side length (l) of the angled elements 120b, base length (h) of the straight elements 120a, inner angle θ, and/or element thickness, to modify the mechanical properties of the cells.

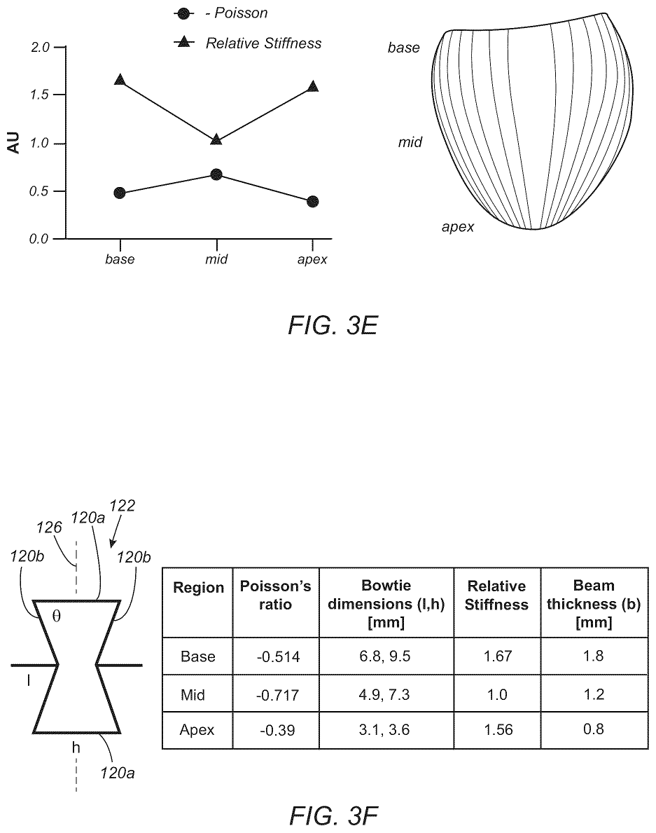

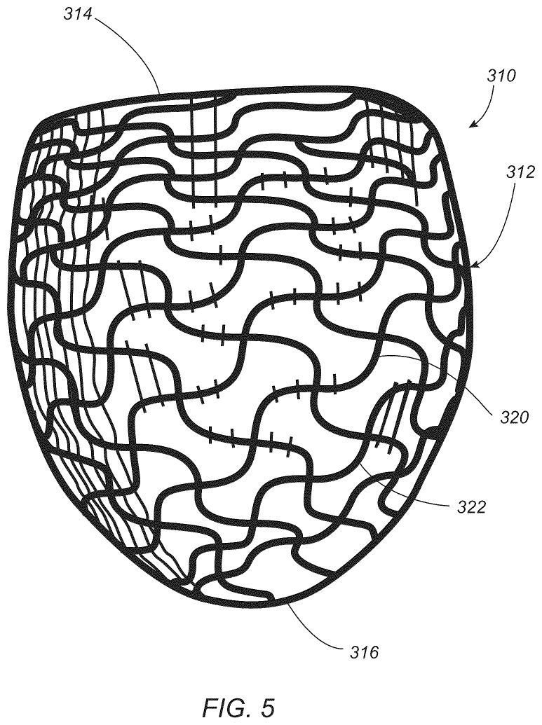

Turning to FIGS. 4A and 4B, yet another example of a device 210 is shown that includes a sleeve 212 generally constructed similar to other devices herein, e.g., principally or entirely from a plurality of elongate elements 220 that are interconnected with one another define a plurality of cells 222 surrounding respective open regions 213. In this example, the cells 222 include a plurality of sinusoidal elements 220 that are interconnected to define the open regions 213. FIG. 5 shows another example of a device 310 that includes a sleeve 312 including a plurality of sinusoidal elements 320 interconnected to define the cells 322 and open regions 313. Although the examples shown include the same configuration of cells (even if the size, shape, width, and/or geometries may be varied in different regions), it will be appreciated that different types of cells may be included in the same support device, if desired.

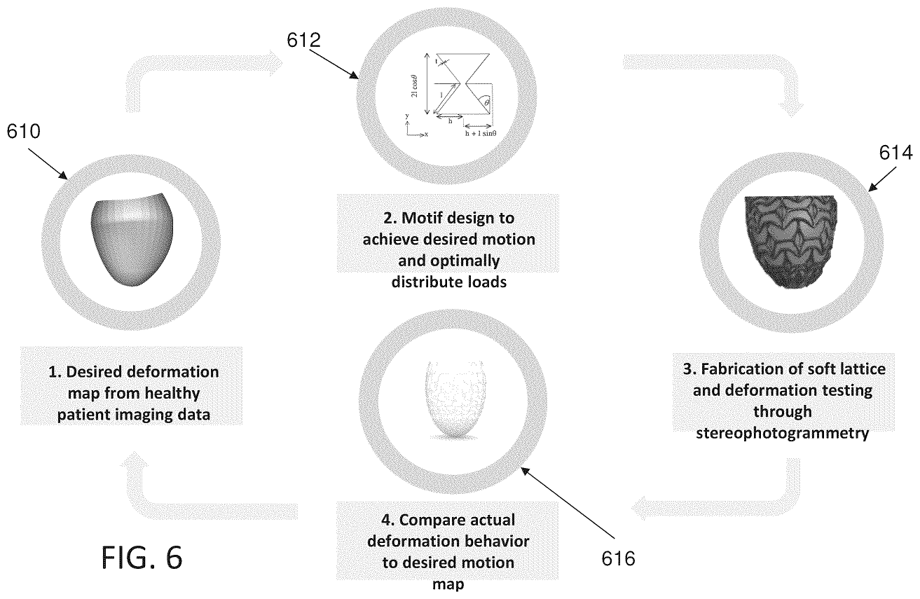

Turning to FIG. 6, an exemplary design process is shown for designing and manufacturing support devices, such as those described above. Generally, the method includes biomimetically replicating complex ventricular structure and motion of a heart, e.g., to mimic and/or replicate normal motion of the heart over which the device is implanted. Thus, in the case of heart fatigue patients, the implanted sleeve device may support the heart to replicate normal motion, which may reduce further deterioration, particularly of the right ventricle.

An iterative design pipeline may be used to fully integrate computational modeling, patient-derived biomechanical data, device design and device evaluation and testing. For example, at step 610, desirable strain distribution maps may be obtained, e.g. from patient-derived imaging data, particularly for the right heart. At step 612, auxetic motifs are rationally and parametrically designed to achieve desired anisotropic behavior. At step 614, desired motifs are parametrically designed and patterned onto lattice structures. Regional variation in material properties (e.g., beam thickness, unit cell density) are incorporated into the design based on the maps obtained at step 610. Sleeve devices may then be manufactured, e.g., by 3D printed using soft, biocompatible materials, such as those described elsewhere herein, and/or using other desired manufacturing methods. Optionally, at step 616, the resulting devices may be mechanically tested and characterized, e.g., using stereophotogrammetry methods. Observed deformation profiles may be comparatively assessed with deformation maps used in step 610 to evaluate mechanical behavior of the lattice of the devices and compared with native tissue response.

Multiple modes of motion contribute to RV pump function, including the shortening of the longitudinal axis and inward movement of the RV free wall. In order to mimic and replicate such complex motion, material selection for the sleeve devices herein should allow for dynamic curvature changes (i.e., convex during end-diastole to flatter at end-systole). Additionally, it may be desirable that sleeve devices substantially match native anisotropic mechanical properties so as to not impede cardiac function of the heart being treated. For example, it may be desirable for the material of the sleeve to expand and contract in orthogonal directions (longitudinal and circumferential) simultaneously and, therefore, auxetic (i.e., possessing a negative Poisson ratio) metamaterial structures, such as those described herein, may achieve the desired biomimetic performance. Auxetic structures exhibit synclastic curvature when subject to out-of-plane bending moments, with geometrically tunable Gaussian curvatures that can be matched to the curved surface of the RV.

The different geometries of cell configurations may provide one or more advantages when included in a support sleeve device. For example, diamond-shaped cells 22, such as those shown in FIGS. 2A and 2B, may provide preferentially longitudinal expansion; bowtie-shaped cells 122, such as those shown in FIGS. 3A-3D, may provide preferentially circumferential expansion; and sinusoidal-shaped auxetic cells 222, such as those shown in FIGS. 4A and 4B, may provide regionally variable properties, e.g., achieved through a density-based gradient from the upper end 214 to the lower end 216. Such regionally heterogeneous material properties may effectively mimic native tissue heterogeneity, thereby enabling improved dynamics and device-tissue coupling.

Returning to FIG. 6, an exemplary manufacturing process for the support devices may include computationally designing unit cells of desired dimensions, which may then be patterned onto 3D and 2D ventricular surfaces generated from de-identified, patient-specific MRI segmentation and/or other analysis of the intended patient. In one method, the device may be cast in a two-step process. First a urethane polymer (e.g., PMC70, SmoothOn, USA) may be cast in a 3D-printed flat mold. After about two to three (2-3) hours from initial casting time, the partially cured lattice structure may be removed from the flat mold and carefully placed on a 3D-printed mandrel shaped after a RV phantom and the edges permanently attached together. This two-step manufacturing process may enable curing of the device in a 3D, stress-free configuration. Taking advantage of the auxetic nature of the cells of the structure, this process may enable a high degree of conformity to the underlying 3D shape.

Optionally, any of the devices herein may include one or more sensors, e.g., for monitoring a patient after the device is implanted over their heart. For example, monitoring heart volume during and after cardiac surgeries may be useful for optimizing survival and gaining insights into response to treatment and real-time volume balancing. If changes in filling volume are noticed early, rapid fluid administration may be used to restore cardiac output. While several options are available for intraoperative volume measurement, fewer are feasible for use in postoperative settings.

Thus, it may be useful to integrate sensing capabilities into the support devices, e.g., to allow substantially continuous volume measurement of the patient. For example, given the convenient placement of the device on the epicardial surface, along with its high degree of conformity to the heart tissue and biomimetic deformation behavior, the device may provide substantially continuous volume measurements by resistive strain sensing.

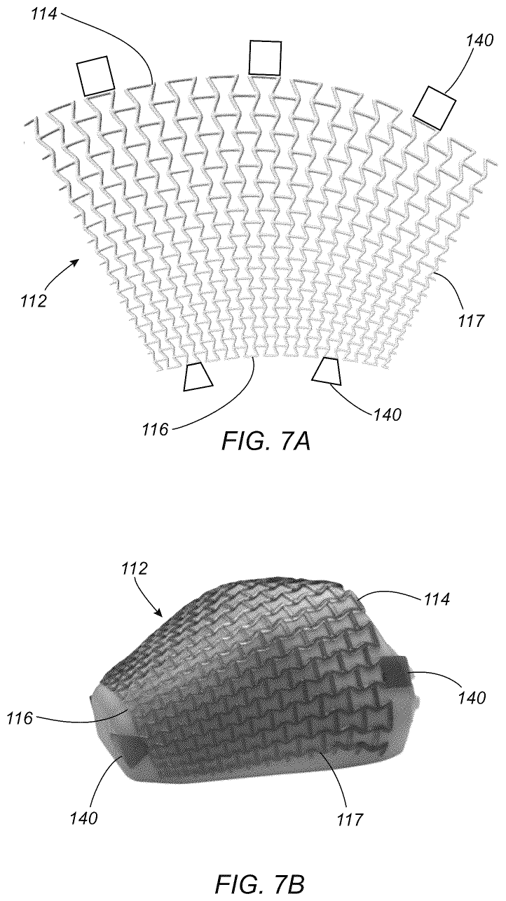

For example, as shown in FIGS. 7A and 7B, nanoparticles may be provided on the outer surface 117 of the sleeve 112, e.g. by spray coating the outer surface 117 with a thin layer, e.g., about forty micrometers (40 μm) thickness of silicone rubber and carbon black nanoparticles. Electrodes may be provided on the sleeve 112 that are coupled to the nanoparticles to provide a resistive strain sensor. For example, as shown, one or more electrodes 140 may be provided on the upper end 114 and lower end 116 of the sleeve 112, and a processor (not shown) may be coupled to the electrodes 140 to acquire signals, which may be processed to correlate the change in resistance to deformation in the myocardium. The processor may be embedded in or otherwise attached to the sleeve 112, e.g., in one of the elements, along with other desired electronic components, e.g., a battery or other power source, memory, and a wireless transmitter for transmitting sensor data to an external device (not shown).

For example, deformation in the myocardium is transferred to the soft strain sensor, which may generate a change in resistance due to the sensor's own deformation, e.g., due the change in cross sectional area and length of the conductive layer. The processor may then process the resulting signals and transmit the signals wirelessly to an external electronic device (not shown), which may store and/or present information related to the deformation to a medical caregiver.

Alternatively, a housing (not shown) may be provided that is separate from the sleeve 112 that contains the electronic components and is coupled to the electrodes 140 by one or more wires or cables (also not shown). The housing may be sized and/or configured to be implanted adjacent the sleeve 112, e.g., subcutaneously within the patient's body to allow monitoring of deformation while facilitating transmission of data to an external electronic device (also not shown). The external device may also include one or more processors, communications interfaces, e.g., wireless receiver and/or transmitter, battery, memory, and/or a display, e.g., such that the external device may receive signals from the implanted processor, and then store and/or display information, e.g., related to deformation of the heart. Thus, the external device may be used to selectively interrogate the implanted processor to acquire data to facilitate monitoring the patient's heart.

Returning to FIG. 1, during use, a sleeve device 10 such as that shown in FIGS. 2A and 2B (or any of the other devices described herein), may be provided including an arrangement of elements 20 coupled together to define a lattice sleeve 12 including an open upper end 14 and a lower end 16. As described above, the configuration of the cells 22 defined by the elements may be customized, if desired, based on the individual anatomy of the patient's heart 90, or one of a standard set of devices may be selected. The device 20 may be positioned over a portion of the patient's heart 90, e.g., overlying at least the right ventricular region. Optionally, the device 10 may be secured to the epicardium 91 of the heart 90, e.g., using one or more of sutures, adhesives, clips or other fasteners (not shown). In addition or alternatively, the inner surface 19 of the sleeve 12 may include materials and/or textures that enhance securing the sleeve 12 relative to the endocardium 91. After implantation, the elements 20 may support the heart 90 indefinitely, e.g., to mimic and/or replicate normal motion of the heart 90, as described elsewhere herein.

Although the devices and methods herein have been described with particular reference to supporting the right ventricle of the heart, it will be appreciated that the devices and methods may be used to treat other cardiac diseases.

In describing representative examples, the specification may have presented the method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for supporting a subject's heart, comprising:
a sleeve configured to be implanted over a region of the subject's heart comprising a lattice defining a surface of the sleeve.

2. The device of claim 1, wherein the lattice comprises auxetic metamaterial.

3-5. (canceled)

6. A passive device for supporting a subject's heart, comprising:
a sleeve comprising an open upper end and a lower end defining an interior region sized to receive a portion of the heart, the sleeve comprising a plurality of elongate elements members coupled together to define a plurality of interconnected cells configured to provide epicardial support of the heart.

7. (canceled)

8. The device of claim 6, wherein the sleeve has an enclosed lower end sized to be positioned over an apex of the heart, wherein the upper end is sized to be received over the epicardium such that the sleeve overlies a right ventricle of the heart.

9. The device of claim 6, wherein the cells are shaped such that the sleeve has anisotropic properties configured to support a right ventricle of the heart under varying loading conditions during a cardiac cycle of the heart.

10. The device of claim 6, wherein the cells are configured to expand and contract to mimic and replicate normal motion of the heart.

11. The device of claim 6, wherein the cells comprise at least one of bowtie shaped cells, diamond-shaped cells, and sinusoidal-shaped cells.

12. The device of claim 6, wherein the cells are shaped such that the sleeve has auxetic properties configured to support a right ventricle of the heart under varying loading conditions during a cardiac cycle of the heart.

13. The device of claim 12, wherein the sleeve comprises a longitudinal axis extending between the upper and lower ends, and wherein the cells are configured to be auxetic orthogonal to the axis.

14. The device of claim 12, wherein the sleeve comprises a longitudinal axis extending between the upper and lower ends, and wherein the cells are configured to be auxetic laterally relative to the axis.

15. The device of claim 12, wherein the cells are configured to be auxetic circumferentially around the sleeve.

16. The device of claim 12, wherein the cells comprise bowtie-shaped cells, each bowtie-shaped cell comprising first and second straight elements opposite one another extending orthogonal to the axis and first and second angled elements extending between opposite ends, respectively, of the first and second straight elements, the angled elements taper inwardly towards one another at intermediate regions between the opposite ends to define the bowtie-shape.

17. The device of claim 6, wherein the cells have different mechanical properties in different regions of the sleeve.

18. The device of claim 17, wherein the cells in a mid-region of the sleeve between the upper and lower ends are configured such that the mid-region has a lower Poisson ratio relative to an upper region adjacent the upper end and a lower region adjacent the lower end.

19. The device of claim 17, wherein the cells in a mid-region of the sleeve between the upper and lower ends are configured such that the mid-region has a higher stiffness relative to an upper region adjacent the upper end and a lower region adjacent the lower end.

20. (canceled)

21. A method for designing a device for supporting a subject's heart, comprising:
generating a desired deformation map from imaging data of a heart;
designing cell parameters for a lattice of the device based at least in part on the deformation map;
forming the device including the lattice from elastic material include cells comprising the cell parameters;
fitting the support device around a phantom heart;
actuating the phantom heart to simulate pulsation of the heart while monitoring deformation of the support device; and
comparing actual deformation of the support device to the desired deformation map.

22-35. (canceled)

36. A method for supporting a subject's heart, comprising:
positioning a sleeve over a region of the heart, the sleeve comprising lattice formed on a surface of the sleeve configured to provide passive support to ventricular tissue of the heart.

37-43. (canceled)

44. The device of claim 6, further comprising a strain sensor array.

45. The device of claim 44, wherein the strain sensor array comprises a nanoparticle coating on an outer surface of the sleeve and one or more electrodes on each of the upper end and the lower end of the sleeve.

46-48. (canceled)

49. The device of claim 44, further comprising a processor coupled to the strain sensor array configured to process signals from the array related to deformation of the heart.

50. (canceled)
